(19) United States
(12) Patent Application Publication
(10) Pub. No.: US 2023/0023413 A1
(43) Pub. Date: Jan. 26, 2023

(54) DEVICE FOR CONTROLLING ENDOSCOPE TO ROTATE

(71) Applicant: Yu Li, Shenzhen (CN)

(72) Inventor: Junjie Wang, Shenzhen (CN)

(21) Appl. No.: 17/662,746

(22) Filed: May 10, 2022

(30) Foreign Application Priority Data
Jul. 26, 2021 (CN) 202121726658.2

(51) Int. Cl. G02B 23/24; G02B 7/02

(52) U.S. Cl. CPC G02B 23/243; G02B 7/02; G02B 23/2461

(57) ABSTRACT

A device for controlling an endoscope to rotate, relating to the technical field of non-destructive inspection, comprises a display device, a control assembly, a coiler and a lens, and further comprises a drive assembly disposed at a joint of the coiler and the lens, and electrically connected to the control assembly to allow users to adjust the angle of the lens by controlling the drive assembly. The drive assembly comprises: a low-speed drive element disposed at an end, away from the display device, of the coiler, and a rotating lever connected to an output shaft of the low-speed drive element. The lens is disposed on a side, away from the low-speed drive element, of the rotating lever. The low-speed drive element is a low-speed motor. The lens comprises a lateral lens and a front lens. The device for controlling an endoscope to rotate has the following advantages: the lens can be rotated to a suitable inspection position without manual operation or other auxiliary location devices, the display angle can be adjusted, using is convenient, and operation is easy.

### CROSS-REFERENCE TO RELATED APPLICATIONS

This application claims the benefit and priority of Chinese Patent Application Number 202121726658.2, filed on Jul. 26, 2021 with China National Intellectual Property Administration, the disclosures of which are incorporated herein by reference in their entireties.

### BACKGROUND OF THE INVENTION

### 1. Technical Field

The invention relates to the technical field of non-destructive inspection, in particular to a device for controlling an endoscope to rotate.

### 2. Description of Related Art

1. Industrial endoscopes are rotated to a suitable angle by swinging a data transmission line or a coiler, the data transmission line or the coiler has to be swing continuously in use, and a video display device should be swung at the same time, which is not beneficial to observation of operators and video collection and storage. Under the enlightenment of domestic surveillance cameras that are able to rotate leftwards and rightwards, the inventor realizes that operators may adjust the angle of an endoscope camera to allow operators to control a motor to rotate forwards or reversely without operating a handle, to drive a rotating device of the industrial endoscopes. However, due to the fact that the endoscope camera is small, a low-speed rotary motor suitable for the small size of the endoscopes needs to be developed and machined to enable an endoscope with a front lens and a lateral lens to rotate under the action of a low-speed rotating device, a gearbox and the motor capable of rotating forward and reversely, so that an operator may rotate or stop the endoscope camera to an optimal observation angle.

2. Most existing industrial endoscopes have limited inspection angles and are rotated a suitable inspection position with manual operation or an auxiliary location device, so operators have to inspect an object closely and cannot find a proper observation position easily if rotating the handle during inspection.

So, a device for controlling an endoscope to rotate is to be studied.

### BRIEF SUMMARY OF THE INVENTION

The objective of the invention is to overcome the defects of the prior art by providing a device for controlling an endoscope to rotate, which has the following advantages: an endoscope lens can be rotated to a suitable inspection position without manual operation or other auxiliary location devices, the display angle can be adjusted, using is convenient, and operation is easy.

The technical solution adopted by the invention to fulfill the above objective is as follows: a device for controlling an endoscope to rotate comprises a display device, a control assembly, a coiler and a lens, and further comprises: a drive assembly disposed at a joint of the coiler and the lens, and electrically connected to the control assembly to allow users to adjust the angle of the lens by controlling the drive assembly.

The drive assembly comprises: a low-speed drive element disposed at an end, away from the display device, of the coiler, and a rotating lever connected to an output shaft of the low-speed drive element, and the lens is disposed on a side, away from the low-speed drive element, of the rotating lever.

The low-speed drive element is a low-speed motor.

The lens comprises a lateral lens and a front lens.

A first fill light is disposed at an end, close to the lateral lens, of the rotating lever, and a second fill light is disposed at an end, close to the front lens, of the rotating lever.

A button is disposed on the control assembly and is made of an insulating material.

Anti-skid lines are disposed on the button.

By adoption of the above technical solution, the invention has the following beneficial effects: the device for controlling an endoscope to rotate comprises a display device, a control assembly, a coiler and a lens, and further comprises: a drive assembly disposed at a joint of the coiler and the lens, and electrically connected to the control assembly to allow users to adjust the angle of the lens by controlling the drive assembly. In this way, the drive assembly effectively overcomes the defects that existing industrial endoscopes have a limited inspection angle and are rotated a suitable inspection position with manual operation or an auxiliary location device, so operators have to inspect an object closely and cannot find out a proper observation position easily if rotating the handle during inspection. The device for controlling an endoscope to rotate has the following advantages: the lens can be rotated to a suitable inspection position without manual operation or other auxiliary location devices, the display angle can be adjusted, using is convenient, and operation is easy.

### BRIEF DESCRIPTION OF THE SEVERAL VIEWS OF THE DRAWINGS

To more clearly explain the technical solutions of the embodiments of the invention or the prior art, drawings used for describing the embodiments of the invention or the prior art will be briefly introduced below. Obviously, the drawings in the following description merely illustrate some embodiments of the invention, and those ordinarily skilled in the art may obtain other drawings according to the following ones without creative labor.

FIG. 1 is a structural diagram of the invention;

FIG. 2 is an enlarged view of part Ain FIG. 1.

Reference signs: 1, display device; 2, control assembly; 3, coiler; 4, drive assembly; 41, low-speed drive element; 42, rotating lever; 43, first fill light; 44, lateral lens; 35, front lens; 46, second fill light.

### DETAILED DESCRIPTION OF THE INVENTION

The invention will be described in further detail below in conjunction with the accompanying drawings.

The specific embodiment is merely used to explain the invention, and is not intended to limit the invention. After reading the specification, those skilled in the art may make non-creative modifications to this embodiment as required within the scope of the claims of the invention, and all these non-creative modifications are protected by patent law.

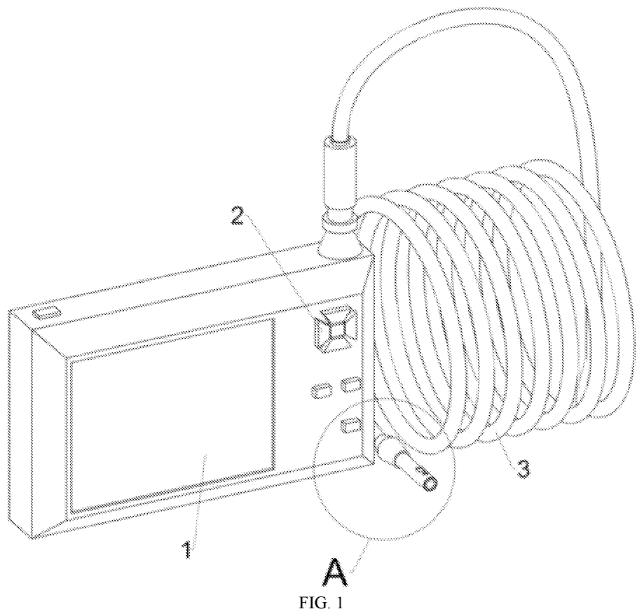

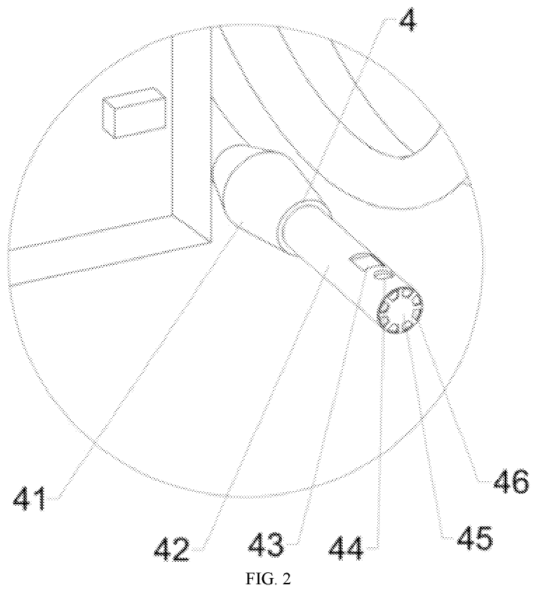

This embodiment relates to a device for controlling an endoscope to rotate, which, as shown in FIG. 1 and FIG. 2, comprises a display device 1, a control assembly 2, a lens, a coiler 3 and a drive assembly 4.

The control assembly 2 is electrically connected to the display device 1 and is composed of a control circuit board and a button; the coiler 3 is electrically connected to the control circuit board, one end of the coiler 3 is electrically connected to the drive assembly 4, and the other end of the coiler 3 is electrically connected to the control assembly 2; the lens is disposed at an end of an output shaft close to the drive assembly 4; and the coiler 3 is electrically connected to the lens. When used, the device is started first, then the lens on the coiler 3 is stretched to a position to be inspected, and transmits captured image information to the control circuit board through the coiler 3, the control circuit board transfers the image information to the display device 1, then a user determines whether the direction of the lens needs to be further adjusted by determining whether an image displayed by the display device 1 is a desired image, at this moment, the user controls the control circuit board to control the drive assembly 4 to rotate forward or reversely through the coiler 3 by pressing different direction buttons; then the drive assembly 4 drives the lens to rotate, so that the angle of the lens is adjusted until an image desired by the user is displayed, thus realizing the inspection of a position that cannot be directly observed with eyes. In this embodiment, the lens comprises a lateral lens 44 and a front lens 45, so that the observation range is wider; the display device 1 is a displayer; and the button is made of an insulating material, and anti-skid lines are disposed on the button. In other embodiments, only one lens, a front lens 45 or a lateral lens, is arranged, and the display device 1 is a mobile phone.

Preferably, as shown in FIG. 2, the drive assembly 4 comprises: a low-speed drive element 41 and a rotating lever 42.

The low-speed drive element 41 is disposed at an end, away from the display device 1, of the coiler 3, the rotating lever 42 is connected to an output shaft of the low-speed drive element 41, the lens is disposed on a side, away from the low-speed drive element 41, of the rotating lever 42, the front lens 45 is disposed at an end, away from the low-speed drive element 41, of the rotating lever 42, the lateral lens 44 is disposed on the rotating lever 42, the direction of the lateral lens 42 is perpendicular to the direction of the front lens 45, a first fill light 43 is disposed at an end, close to the lateral lens 44, of the rotating lever 42, and a second fill light 46 is disposed at an end, close to the front lens 45, of the rotating lever 42. In this embodiment, the low-speed drive element 41 is a low-speed motor.

The working principle of the invention is approximately as follows: the control assembly 2 is electrically connected to the display device 1 and is composed of the control circuit board and the button; the coiler 3 is electrically connected to the control circuit board, one end of the coiler 3 is electrically connected to the drive assembly 4, and the other end of the coiler 3 is electrically connected to the drive assembly 4; the lens is disposed at one end of the output shaft close to the drive assembly 4; the coiler 3 is electrically connected to the lens; when used, the device is started first, then the lens on the coiler 3 is stretched to a position to be inspected, and transmits captured image information to the control circuit board through the coiler 3, the control circuit board transfers the image information to the display device 1, then a user determines whether the direction of the lens needs to be further adjusted by determining whether an image displayed by the display device 1 is a desired image, at this moment, the user controls the control circuit board to control the drive assembly 4 to rotate forward or reversely through the coiler 3 by pressing different direction buttons; then the drive assembly 4 drives the lens to rotate, so that the angle of the lens is adjusted until an image desired by the user is displayed, thus realizing the inspection of a position that cannot be directly observed with eyes. In this way, the drive assembly 4 effectively overcomes the defects that existing industrial endoscopes have a limited inspection angle and are rotated a suitable inspection position with manual operation or an auxiliary location device, so operators have to inspect an object closely and cannot find out a proper observation position easily if rotating the handle during inspection. The device for controlling an endoscope to rotate has the following advantages: the lens can be rotated to a suitable inspection position without manual operation or other auxiliary location devices, the display angle can be adjusted, using is convenient, and operation is easy.

The above embodiment is merely used to explain, rather than limiting, the technical solutions of the invention. Other amendments or equivalent substitutions made according to the technical solutions of the invention without departing from the spirit and scope of the invention should also fall within the scope of the claims of the invention.

What is claimed is:

1. A device for controlling an endoscope to rotate, comprising a display device (1), a control assembly (2), a coiler (3) and a lens, wherein the device for controlling an endoscope to rotate further comprises: a drive assembly (4) disposed at a joint of the coiler (3) and the lens, and electrically connected to the control assembly (2) to allow users to adjust an angle of the lens by controlling the drive assembly (4).

2. The device for controlling an endoscope to rotate according to claim 1, wherein the drive assembly (4) comprises: a low-speed drive element (41) disposed at an end, away from the display device (1), of the coiler (3), and a rotating lever (42) connected to an output shaft of the low-speed drive element (41), and the lens is disposed on a side, away from the low-speed drive element (41), of the rotating lever (42).

3. The device for controlling an endoscope to rotate according to claim 2, wherein the low-speed drive element (41) is a low-speed motor.

4. The device for controlling an endoscope to rotate according to claim 2, wherein the lens comprises a lateral lens (44) and a front lens (45).

5. The device for controlling an endoscope to rotate according to claim 4, wherein a first fill light (43) is disposed at an end, close to the lateral lens (44), of the rotating lever (42), and a second fill light (46) is disposed at an end, close to the front lens (45), of the rotating lever (42).

6. The device for controlling an endoscope to rotate according to claim 1, wherein a button is disposed on the control assembly (2) and is made of an insulating material.

7. The device for controlling an endoscope to rotate according to claim 6, wherein anti-skid lines are disposed on the button.
